(19) United States
(12) Patent Application Publication
(10) Pub. No.: US 2013/0289023 A1
(43) Pub. Date: Oct. 31, 2013

(54) METHOD FOR TREATING BRAIN TUMOR

(71) Applicants: Chi-Ying Huang, Taipei City (TW); Yu-Hsin Liang, Taipei City (TW); Hui-Wen Cheng, Taichung City (TW)

(72) Inventors: Chi-Ying Huang, Taipei City (TW); Yu-Hsin Liang, Taipei City (TW); Hui-Wen Cheng, Taichung City (TW)

(21) Appl. No.: 13/873,405

(22) Filed: Apr. 30, 2013

Related U.S. Application Data
(60) Provisional application No. 61/640,363, filed on Apr. 30, 2012.

(51) Int. Cl. A61K 31/5415; A61K 45/06

(52) U.S. Cl. CPC A61K 31/5415; A61K 45/06
USPC 514/225.5; 514/225.8; 514/226.2

(57) ABSTRACT

The preset invention relates to a new method for treating brain tumor with an antipsychotic phenothiazine derivative as a brain tumor cell inhibitor or a brain tumor stem cell inhibitor. In particular, an antipsychotic phenothiazine is accessible to brain via blood-brain barrier, which should be beneficially in treatment of brain tumor.

### CROSS REFERENCE TO RELATED APPLICATION

This application claims priority of U.S. Provisional Application No. 61/640,363 filed on Apr. 30, 2012. The content of the application is incorporated herein by reference in its entirety.

### FIELD OF THE INVENTION

The present invention relates to a new method for treating brain tumor.

### BACKGROUND OF THE INVENTION

Brain tumors include all tumors inside the cranium or in the central spinal canal, including primary brain tumors that are created by an abnormal and uncontrolled cell division, usually in the brain itself, and metastatic brain tumors that have spread to the brain from another location in the body. Any brain tumor is inherently serious and life-threatening because of its invasive and infiltrative character in the limited space of the intracranial cavity. Brain tumors or intracranial neoplasms can be cancerous (malignant) or non-cancerous (benign); however, the definitions of malignant or benign neoplasms differs from those commonly used in other types of cancerous or non-cancerous neoplasms in the body. Its threat level depends on the combination of factors like the type of tumor, its location, its size and its state of development. Because the brain is well protected, by the skull, the early detection of a brain tumor only occurs when diagnostic tools are directed at the intracranial cavity. Usually detection occurs in advanced stages when the presence of the tumor has caused unexplained symptoms. Among brain tumors, glioblastoma (GBM) is an aggressive tumor with a poor prognosis.

Not all chemotherapy drugs are suitable for treating brain tumors because some chemotherapy drugs cannot cross the natural protection filter around the brain (i.e., the blood-brain barrier). Currently, only Temozolomide has been approved by the Food and Drug Administration (FDA), U.S.A. for GBM treatment. Therefore, there is an urgent need to discover candidate therapeutic drugs for brain tumors, particularly GBM.

### SUMMARY OF THE INVENTION

The invention provides a new method for treating a brain tumor with anti-psychotic drugs that are accessible to brain through the blood-brain barrier, which should be beneficial in the treatment of a brain tumor.

In one aspect, the invention provides a method for treating a brain tumor in a subject comprising administering to the subject a therapeutically effective amount of an antipsychotic phenothiazine derivative.

In some examples of the invention, the antipsychotic phenothiazine derivative is selected from the group consisting of thioridazine, acepromazine, fluphenazine, perphenazine, prochlorperazine, promazine, promethazine, trifluoperazine, triflupromazine and chlorpromazine. In particular samples of the invention, the compound is thioridazine, fluphenazine, trifluoperazine and prochlorperazine. A preferred example is thioridazine. Another preferred example is fluphenazine.

In the invention, the brain tumor includes primary brain tumors and metastatic brain tumors. In one example of the invention, the brain tumor is glioblastoma (GBM).

In another aspect, the invention provides a method for inhibiting the growth of brain cancer stem cells in a subject comprising administering to the subject a therapeutically effective amount of an antipsychotic phenothiazine derivative.

The details of one or more embodiments of the invention are set forth in the description below. Other features, objects, and advantages of the invention will be apparent from the description and from the claims.

### BRIEF DESCRIPTION OF THE DRAWINGS

The foregoing summary, as well as the following detailed description of the invention, will be better understood when read in conjunction with the appended, drawing. In the drawings;

FIG. 1 provides the effects on the cell viability of brain tumor cells treated with various anti-psychotic drugs in GBM cell lines; including:

FIG. 1(A) provides the results of the MTT assay for determining cell viability after treatment with 10 μM various anti-psychotic drugs in GBM8401.

FIG. 1(B) shows the effects of thioridazine, fluphenazine, and temozolomide on the cell viability after the treatments of 1, 5, 10 μM thioridazine, fluphenazine, and temozolomide (the only FDA approved drag for GBM, as a positive control) for 24 hr in GBM8401 (upper) and U87MG (lower) cells, respectively: and the results of the clonogenic assay for determining cell viability after treatment with thioridazine at the concentrations of 0 μM, 1 μM, 333 μM and 10 μM in GBM8401 cells.

FIG. 2 provides the effects of thioridazine on inducing autophagy; including

FIG. 2(A) provides the results of the treatment of thioridazine at the concentrations of 5 μM, 10 μM, 15 μM, respectively, for 24 hours, in which the cell lysates were subjected to western blot analysis, and the PI3K, phospho-mTOR (Ser 2448), phospho-S6K (Ser 424) were down-regulated, whereas phospho-AMPK (Thr 172) and LC-3 II were up-regulated.

FIG. 2(B) shows that the PI3K was down-regulated in GBM8401 and phospho-Akt (Ser 473) was down-regulated in U87MG.

FIG. 2(C) shows that ER stress marker IRE1α, Bip and CHOP were up-regulated.

FIG. 2(D) provides the results of the treatment of fluphenazine at the concentrations of 5, 10, 15, 20 μM, respectively, for 24 hours.

FIG. 3 provides the effects of thioridazine, fluphenazine, trifluoperazine, and prochlorperazine on cell viability of U87MG sphere cells; including:

FIG. 3(A) shows that U87MG sphere cells were treated with 10 and 20 μM thioridazine for 24 hours, respectively; and the cell viability of U87MG sphere cells was determined by counting cells with trypan blue,

FIG. 3(B) shows that U87MG sphere cells were treated with 10 and 20 μM fluphenazine for 24 hours, respectively.

FIG. 3(C) shows that U87MG sphere cells were treated with 10 and 20 μM trifluoperazine tor 24 hours, respectively.

FIG. 3(D) shows that U87MG sphere cells were treated with 10 and 15 μM Prochlorperazine for 24 hours, respectively.

FIG. 3(E) shows that the cancer stem-like cells population was significantly decreased by treatment with thioridazine (10 μM) from 1.26% to 0.03% in GBM8401 cells and from 1.43% to 0.1% in U87MG cells, as determined by side population assay.

FIG. 4 provides the pathways analysis of differentially expressed gene signatures using microarray profiling, including:

FIG. 4(A) shows that the up- and down-regulated gene lists alter 10 μM Thioridazine treatment from connectivity map database and the microarray data, from GBM8401.

FIG. 4(B) shows that the prioritized pathways as analyzed via CPDB pathway analyzer included pathway in cancer, G-protein couple receptor protein signaling, senescence and autophagy, DMA damage response, and G1 to S cell cycle control.

FIG. 4(C) shows that to validate the microarray dataset, GBM8401 and U87MG were treated with 10 μM Thioridazine for 24 hours, respectively; and the cell lysates were subjected to western blot analysis using Endothelin receptor type A (ENDRA), GPR17, GPRC5B antibody, the target involved in G-protein couple protein signaling.

FIG. 5 shows the results of thioridazine on suppression of GBM tumorigenesis in vivo; including;

FIG. 5(A) shows that U87MG cells (1×10⁶ cells/injection) were subcutaneously implanted into NOD/SCID mice and subdivided into two groups; control (DMSO) and thioridazine (5 mg/kg/day, 5 days/week ); the tumor size was measured using caliper on a weekly basis.

FIG. 5(B) provides the representative photographs of tumor biopsies obtained from control and thioridazine treated animals.

### DETAILED DESCRIPTION OF THE INVENTION

Unless defined otherwise, all technical and scientific terms used herein have the same meaning as commonly understood by a person skilled in the art to which, this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

The term “brain tumor” as used herein refers to any tumor inside the cranium or in the central spinal canal, including primary brain tumors, and metastatic brain tumors. The term “primary brain tumor” as used herein refers to a tumor that arises in the brain. The term “metastatic brain tumor” as used herein refers to a tumor that spreads from another area of the body through the bloodstream to the brain.

As used herein, the term “subject” refers to any organism suffering from a brain tumor, encompassing humans or non-human mammals or animals. Non-human mammals include livestock animals, companion animals, laboratory animals,, and non-human primates. Non-human subjects also include, without limitation, horses, cows, pigs, goats, dogs, cats, mice, rats, guinea pigs, gerbils, hamsters, mink, rabbits and fish. It is understood that the preferred subject is a human, in some embodiments of the invention, the term “subject” refers to a biological sample as defined herein, which includes but is not limited to a cell, tissue, or organ that is isolated from a human or non-human subject suffering from a brain tumor. Accordingly, the methods disclosed herein are intended to be applied in vivo as well as m vitro.

As used herein, the term “therapeutically effective amount” refers to an amount sufficient for providing an effect in treatment for a brain tumor, which is depending on the mode of administration and the condition to he treated, including age, body weight, symptom, therapeutic effect, administration route and treatment time.

As used herein, the term “antipsychotic phenothiazine derivative” or “anti-psychotic drug” refers to phenothiazine, having the formula of S(C₆H₄)₂NH, and its derivative, which is used as an antipsychotic drug. Phenothiazine is related to one of the thiazine-class of heterocyclic compounds. Its derivatives refer to a group of 10H-phenothiazine derivatives bearing an alkyl substituent, a halogen-containing group such as a halogen or a haloalkyl or a sulfur-containing group. It is known that antipsychotic phenothiazine derivatives are accessible to brain through the blood-brain barrier, and thus they should be beneficial in treatment of a brain tumor. According to the examples of the invention, the antipsychotic phenothiazine derivatives include but are not limited to thioridazine, acepromazine, fluphenazine, perphenazine, prochlorperazine, promazine, promethazine, trifluoperazine, triflupromazine and chlorpromazine, whose structures are shown in Table 1. In particular samples of the invention, the antipsychotic phenothiazine derivative is thioridazine, fluphenazine, trifluoperazine or prochlorperazine. One preferred example is thioridazine, which is commonly used in humans as recorded in National Health Insurance Research Database and can cross through blood-brain barrier. Another preferred example is fluphenazine.

TABLE 1
Anti-psychotic drugs

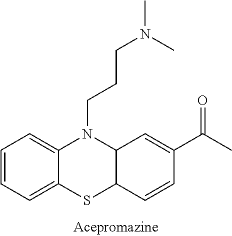 [SMILES: CC(=O)C1=CC2C(C=C1)Sc1ccccc1N2CCCN(C)C]

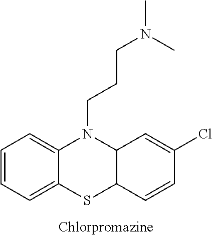 [SMILES: CN(C)CCCN1c2ccccc2SC2C=CC(Cl)=CC21]

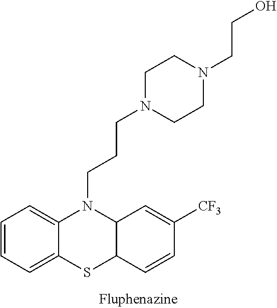 [SMILES: OCCN1CCN(CCCN2c3ccccc3SC3C=CC(C(F)(F)F)=CC32)CC1]

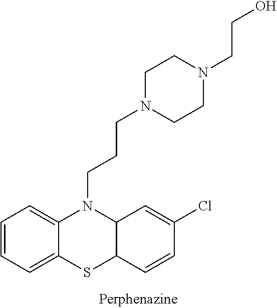 [SMILES: OCCN1CCN(CCCN2c3ccccc3SC3C=CC(Cl)=CC32)CC1]

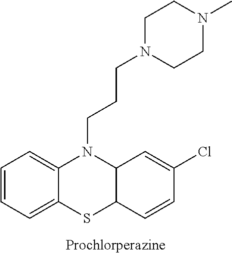 [SMILES: CN1CCN(CCCN2c3ccccc3SC3C=CC(Cl)=CC32)CC1]

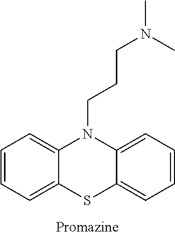 [SMILES: CN(C)CCCN1c2ccccc2Sc2ccccc21]

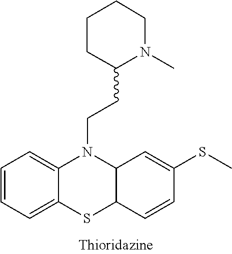 [SMILES: CSC1=CC2C(C=C1)Sc1ccccc1N2CCC1CCCCN1C]

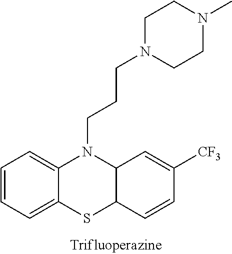 [SMILES: CN1CCN(CCCN2c3ccccc3SC3C=CC(C(F)(F)F)=CC32)CC1]

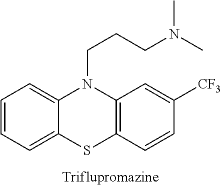 [SMILES: CN(C)CCCN1c2ccccc2Sc2ccc(C(F)(F)F)cc21]

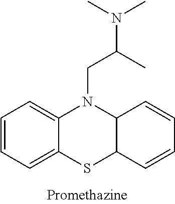 [SMILES: CC(CN1c2ccccc2SC2C=CC=CC21)N(C)C]

In this invention, it was hypothesized that if a drug signature could at least partially reverse the gene expression signature of GBM, it might have the potential to inhibit GBM-related pathways and thereby treat GBM. It was evaluated that the in vitro anti-tumor effects of 108 potential drugs via MTT cell viability assays and clonogenic assays in GBM cell lines, it was unexpectedly found in this invention that an antipsychotic phenothiazine derivative, such as thioridazine, fluphenazine, trifluoperazine or prochlorperazine, was effective in treatment of a brain tumor, particularly GBM.

In the previous studies, GBM cells were shown to be more sensitive to agents that induce autophagy than apoptosis. However, it was unexpectedly found in the invention that the treatment of an anti-psychotic drug in GBM cells did not trigger apoptosis. in one example of the invention, the treatment of thioridazine could result in PI3K, phospho-mTOR, and phospho-S6K down-regulation, whereas phospho- AMP& and LC-3 II were up-regulated, suggesting that thioridazine could induce autophagy in GBM cells. In addition, GBM cancer stem-like cells have been proposed to be involved in GBM resistance and recurrence, and our bioinformatics analysis indicates that thioridazine can also partially reverse the gene signatures of cancer stem-like cell. In agreement with the prediction, thioridazine inhibited the formation of primary GBM spheroids. Finally, using microarray profiling and western blot analysis, thioridazine might target to G-protein. couple receptor (GPCR)-mediated pathway. In conclusion, an anti-psychotic drug, such as Thioridazine, Fluphenazine, Trifluoperazine or Prochlorperazine, can be used as a potent anti-GBM agent.

Accordingly, the invention provides a method for treating a brain tumor in a subject comprising administering to the subject a therapeutically effective amount of an antipsychotic phenothiazine derivative.

In one example of the invention, the brain tumor is glioblastoma (GBM).

On the other hand, the invention provides a method for inhibiting the growth of brain cancer stem cells in a subject comprising administering to the subject a therapeutically effective amount of an antipsychotic phenothiazine derivative.

The invention also provides the use of an antipsychotic phenothiazine derivative for manufacturing a medicament for treating a brain tumor, particularly GBM.

According to the invention, the antipsychotic phenothiazine derivative may be formulated and administered in a pharmaceutical composition in any route that is appropriate, including but not limited to parenteral or oral administration. The pharmaceutical compositions for parenteral administration include solutions, suspensions, emulsions, and solid injectable compositions that are dissolved or suspended in a solvent immediately before use. The injections may he prepared by dissolving, suspending or emulsifying one or more of the active ingredients in a diluent. Examples of said diluents are distilled water for injection, physiological, saline, vegetable oil, alcohol, and a combination thereof. Further, the injections may contain stabilizers, solubilizers, suspending agents, emulsifiers, soothing agents, buffers, preservatives, etc. The injection is sterilized in the final formulation step or prepared by sterile procedure. The pharmaceutical composition of the invention may also be formulated into a sterile solid preparation, for example, by freeze-drying, and may be used after sterilized or dissolved in sterile injectable water or other sterile diluent(s) immediately before use.

According to one example of the invention, the pharmaceutical composition of the antipsychotic phenothiazine derivative is orally administered in a solid or liquid form. Examples of the solid form of the oral composition include tablets, pills, capsules, dispersible powders, granules, and the like. The oral compositions also include gargles which are to be stuck to oral cavity and sublingual tablets. The capsules include hard capsules and soft capsules. In such solid compositions for oral use, one or more of the active compound(s) may be admixed solely or with diluents, binders, disintegrators, lubricants, stabilizers, solubilizers, and then formulated into a preparation in a conventional manner. When necessary, such preparations may be coated with a coating agent, or they may be coated with two or more coating layers. On the other hand, the oral compositions in a liquid form include pharmaceutically acceptable aqueous solutions, suspensions, emulsions, syrups, elixirs, and the like. In such compositions, one or more of the active compound(s) may be dissolved, suspended or emulsified in a commonly used diluent (such as purified water, ethanol or a mixture thereof etc.). Besides such diluents, said compositions may also contain wetting agents, suspending agents, emulsifiers, sweetening agents, flavoring agents, perfumes, preservatives and buffers and the like.

The invention also provides a method for treating glioblastoma in a combination therapy. According to the invention, the antipsychotic phenothiazine derivative may he administered to the subject, in combination with another therapeutic drug for treating a brain tumor such as glioblastoma. In one example of the invention, another therapeutic drug for treating a brain tumor is a chemotherapy drug. For a combination therapy, the antipsychotic phenothiazine derivative may be formulated as a composition containing other therapeutically active ingredients in a single dose form and/or a kit containing the antipsychotic phenothiazine derivative and other therapeutically active ingredients individually in separate dose forms. The active ingredients used in combination therapy may be co-administered or administered separately.

The following examples are given for the purpose of illustration (July and are not intended to limit the scope of the invention.

### EXAMPLE

Material and Method

Clonogenic Assay

The GBM cancer cell line, GBM8401 was maintained in a DMEM medium. GBM8401 cells were seeded respectively in 6 well plates with 10³ cells per well for 14 days. Each well contained 2 ml DMEM medium as cultured condition for GBM8401 cells. Thioridazine (Sigma), fluphenazine (Sigma) or other chemicals was added 24 hours after seeding of the cells. The medium and Thioridazine were changed once on day 4. After the treatments, cells were washed with PBS, and the colonies were fixed with fix solution (acetic acid: methanol=1:3) and stained with 0.5% crystal violet in methanol. After removing the crystal violet carefully and rinse with tap water, the colonies were counted manually.

Tumor Spheroid Assay

For the formation of tumor spheroids, cells were cultured in HEScGRO serum-free medium (human) (Chemicon) supplemented with 20 ng/mL Hegf, 10 ng/mL hFGF-b and NeuroCult MS-A proliferation supplements. Cells were seeded at low densities (1000 cells/mL) in 12-well low adhesion plates at 1 mL, per well Spheroids (tight, spherical, non-adherent masses >90 um in diameter) were counted, and at least 50 spheroids per group were measured with an ocular micrometer. For secondary spheroid-forming assays, primary spheroids were dissociated mechanically and processed as in the primary assay. For the quantification of the percentage of spheroid-forming cells, cells were seeded at one cell per well in 96-well plates,

Side Population Analysis and Purification Using Flow Cytometry

GBM8401 and U87MG cells were treated with thioridazine at 5 μM and 10 μM or fluphenazine at 5 μM and 10 μM for 24 hours. The cells were detached from the dishes with trypsin-EDTA (Invitrogen), and suspended in a single-cell suspension at 1×10⁶ cells/mL in Hank's balanced salt solution (HBSS) supplemented with 3% fetal calf serum and 10 mM Hepes. The cells were then incubated at 37° C. for 90 min with 20 μg/mL Hoechst 33342 (Sigma Chemical, St. Louis, Mo.), either alone or in the presence of 50 μmol/L verapamil (Sigma), an inhibitor of the verapamil-sensitive ABC transporter. After incubation, the cells were centrifuged immediately for 5 min at 300 g and 4° C. and resuspended in ice-cold HBSS, The cells were kept on ice to inhibit efflux of the Hoechst dye, and 1 μg/mL propidium Iodide (BD) was added to discriminate dead cells. Finally, these cells were filtered through a 40 μm cell strainer (BD) to obtain a single-cell suspension. Dual-wavelength analysis and purification were performed on a dual-laser PACS. Vantage SE machine (BD). Hoechst 33342 was excited with a 355 nm UV light and emitted blue fluorescence with a 450/20 band-pass (BP) filter and red fluorescence with a 675 nm edge filter long-pass (EFLP). A 610 nm dichroic mirror short-pass (DMSP) was used to separate the emission wavelengths. Pi-positive (dead) cells were excluded from the analysis.

Western Blotting Analysis

The cells were lysed in a lysis buffer (50 mM Tris-HCl, pH 7.4, 5 mM MgCl₂, 1% Nonidet P-40, 150 mMNaCl, 1 mM phenylmethylsulfonyl fluoride). Total protein was isolated and subjected to SDS polyacrylamide gel electrophoresis and electrotransfered onto PVDF membranes (Millipore). Primary antibodies, including p-mTOR (Ser 2448), mTOR, PI3K (P110α), p-Akt (Ser 473), Akt, p-S6K (Ser 424), S6K, p-AMPK (Thrl72), AMPK, and CASP3, were obtained from Cell Signaling and LC3 was from Abgent, and secondary antibodies for anti-mouse and anti-rabbit horseradish preoxidase (HRP)-conjugation were from Chemicon International. The protein detection was performed with enhanced chemiluminescence (ECL™) method captured by a Luminescence Imaging System (LAS-4000™, Fuji Photo Film Co., Ltd),

In Vivo Examination of the Tumor Suppressing Effects by Thioridazine Treatment

Female NOD/SCID, 4-6 weeks of age were purchased from the National Laboratory Animal Center (NLAC) (Taipei, Taiwan). All protocols were approved by the institutional animal care committee of Taipei Medical University Hospital Mice were inoculated with U87MG cells in the right flank of NOD/SCID mice (1×10⁶ cells/100 μL PBS, subcutaneous infection) and divided into control (sham) and thioridazine-treatment groups (5 mg/kg/day, 5 days/week). N=5 in each group. Tumor burden was measured on a weekly basis using a caliper and calculated using the following formula (a×b²)/2, where a and b represents the long and short axis of the tumor respectively.

Results

Analysis of Cell Viability After Treating Anti-Psychotic Drugs in GBM Cell Lines

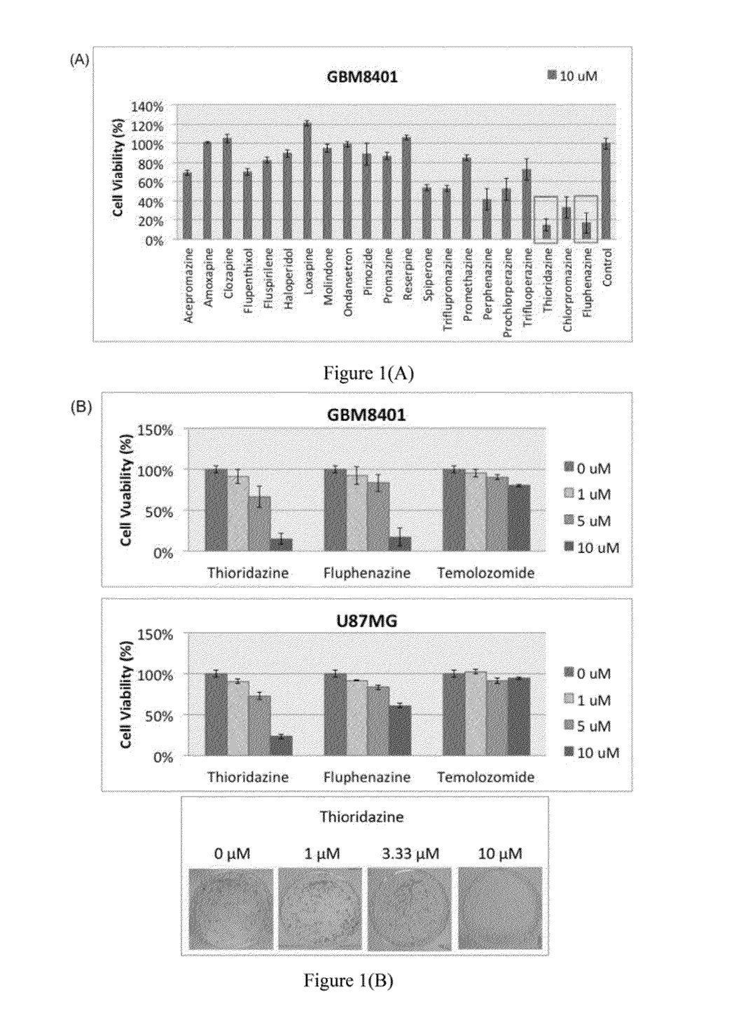

To examine the cytotoxicity of anti-psychotic drugs on malignant glioma, GBM8401 cells were treated with 21 anti-psychotic drugs at 10 μM, respectively, for 72 hr. As shown in FIG. 1(A), four drags (perphenazine, thioridazine,, chlorpromazine, and fluphenazine) had IC₅₀ value <10 μM. In particular, thioridazine and fluphenazine were more effective in cytotoxicity. Subsequently, two malignant glioma cell lines (GBM8401 and U87MG) were treated with thioridazine and fluphenazine at concentrations ranging from 1 to 10 μM for 72 hr and a dose-dependent increase in cell death was observed, see FIG. 1(B).

Effects of Thioridazine Inducing Autophagy and ER Stress

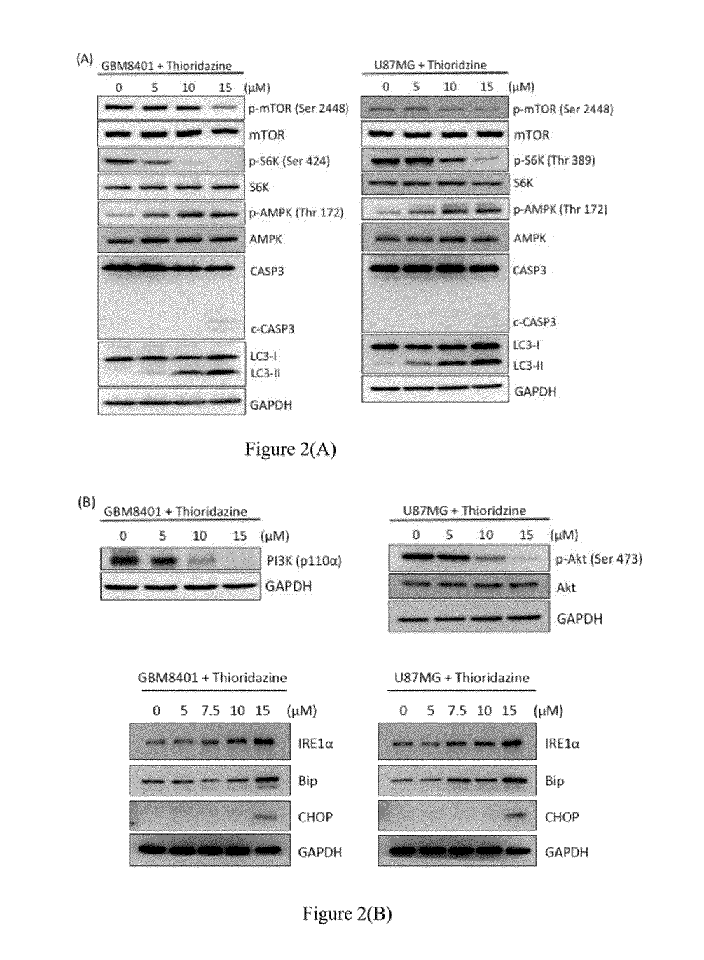

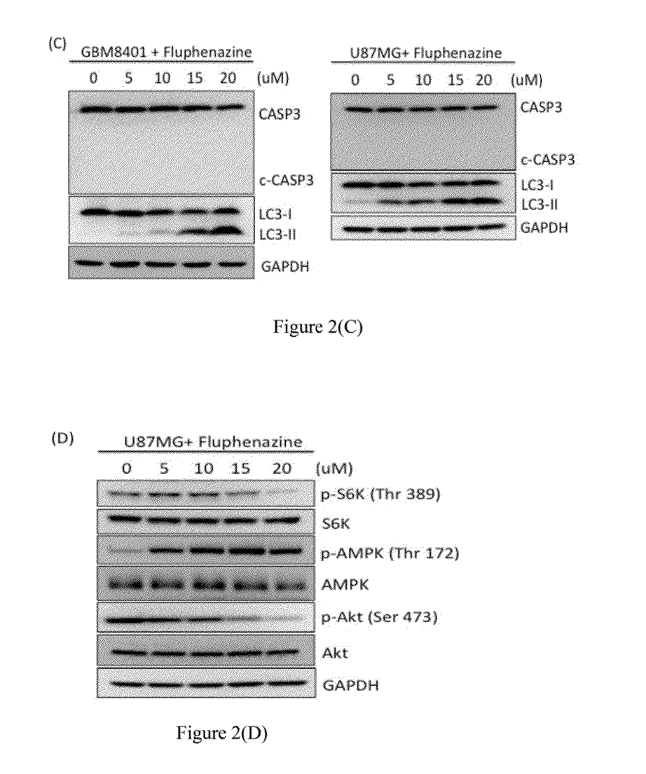

To investigate the mechanism underlying the cytocidal effect of thioridazine, GBM8401 cells were treated with 5 μM thioridazine, and examined the protein expressions by microwestern, which enable quantitative, sensitive and high-throughput (96 different antibodies) assessment of proteins. We observed several protein expression changes, primarily involved in autophagic and cell growth related pathway, it was shown that GBM cells are more sensitive to agents that induce autophagy than apoptosis. Thus, we examined the PI3K-Akt-mTORcascade with known roles m regulation of autophagy. GBM8401 and U87MG cells were treated with Thioridazine at concentrations ranging from 5 to 15 μM for 24 hr. The result showed that PI3K, phospho-Akt(Ser 473), phospho-mTOR(Ser 2448), phospho-S6K (Ser 424) were down-regulated, whereas LC3-II, a specific marker of autophagy, and phospho-AMPK (THr172) were up-regulated (FIG. 2(A,B)). Similar results were also observed, when GBM8401 and U87MG cells were treated with Fluphenazine (FIGS. 2C and D). These data suggest that both thioridazine and fluphenazine induce autophagye in GBM cells. At the same time, the induction of EE stress in GBM cells by thioridazine was evidenced by detection of unfolded protein response activation (accumulation of IRE1a) and accumulation of ER stress-associated proteins (Bip and CHOP) (the lower of FIG. 2B). It showed that ER stress could be the upstream signal of autophagy.

Effects of Thioridazine and Fluphenazinein Cell Viability of U87MG Sphere Cells.

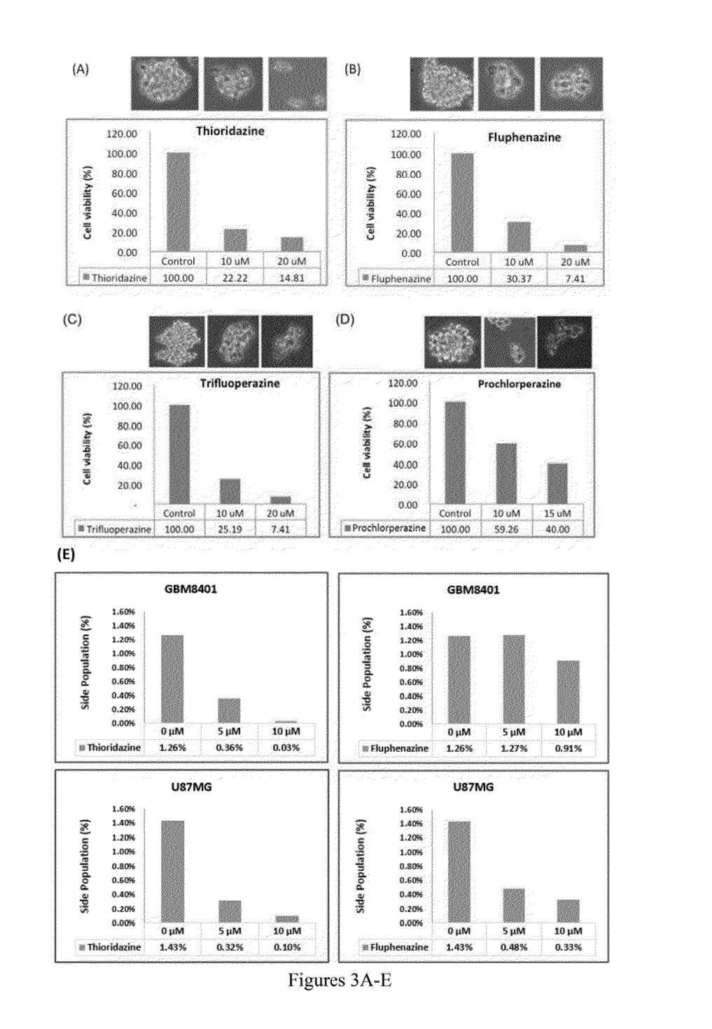

To Investigate whether thioridazine and fluphenazine had anti-cancer stem-like cell property, U87MG sphere cells (cancer stem-like cells) were treated with thioridazine (FIG. 3(A)), fluphenazine (FIG. 3(B)), and trifluoperazine (FIG. 3(C)) at 10 and 20 μM and prochlorperazine (FIG. 3(D)) at 10 and 15 μM for 24 hr, respectively. The cell viability was significantly reduced. For example, the cell viability was reduced to 22% when U87MG sphere cells were treated with 10 μM thioridazine.

Pathways Analysis of Differentially Expressed Gene Signatures Using Microarray Profiling

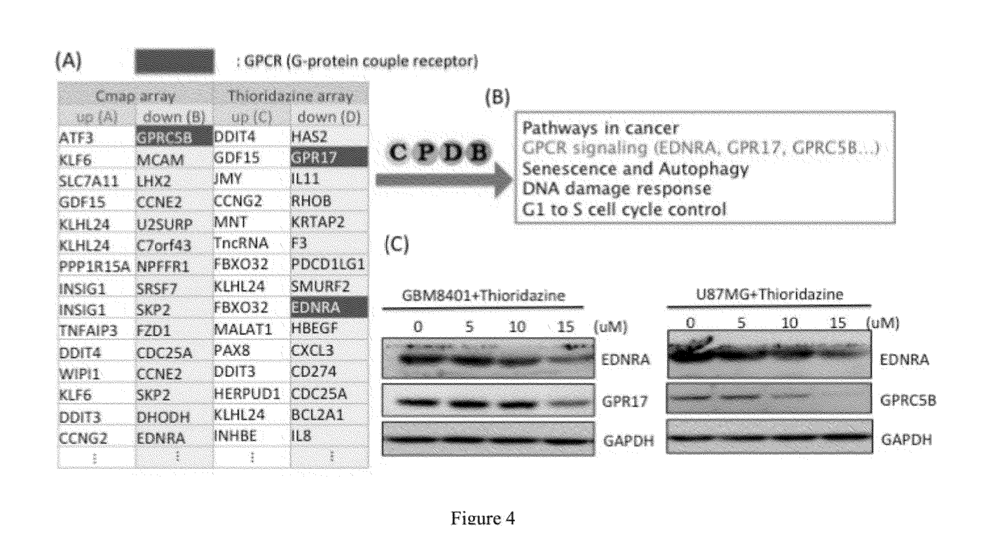

To identify the target of thioridazine in treating GBM, GBM8401 cells were treated with 10 μM thioridazine for 24 hr, and then subjected to microarray profiling. Pathway analysis of differentially expressed gene signatures obtained from microarray profiling was investigated via Consensus PathDB. Several thioridazine-mediated pathways were highlighted, including pathway in cancer, GPCR signaling, senescence and autophagy, DMA damage response, and G1 to S cell cycle control (FIGS. 4(A) and 4(B)). Because G-protein couple receptors (GPCR) are membrane associated proteins and have been widely recognized as drug targets. Therefore, we tested whether GPCR might be the target of thioridazine. GBM8401 and U87MG cells were treated with 10 μM thioridazine for 24 hr and the lysates were subjected to western blotting. As shown in FIG. 4(C), EDNRA, GPR17, GPRC5B, three of the GPCR were down-regulated after the treatment of thioridazine, suggesting that thioridazine-elicited pathways might be modulated via GPCR.

Thioridazine Suppressed Tumorigenesis of U87MG Cells in Immune Compromised Mice

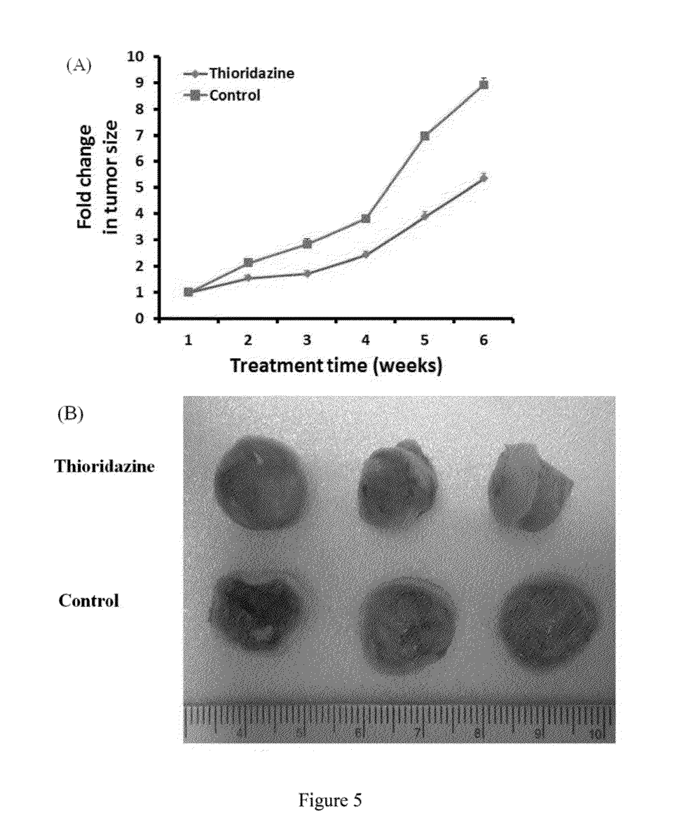

The anti-tumor effect of thioridazine was validated in vivo using immune compromised NOD-SCID mice which were inoculated with U87MG cells. Mice received the treatment, with thioridazine (i.p. injection 5 mg/kg/day, 5 days/week.) exhibited a significantly smaller tumor burden as compared to those with vehicle control (see FIG. 5(A)). Three weeks post treatment, a significantly difference in tumor size between thioridazine-treated and control mice was observed. By the end of 6 weeks, animals were humanely sacrificed and tumor biopsies were collected. Evidently, thioridazine-treated tumor samples were significantly smaller in size and less vascularized as compared to those from the control mice (see FIG. 5(B)). These in vivo observations agreed with our in vitro data that, thioridazine effectively suppressed the tumorigenesis of U87MG cells.

It was concluded that the anti-psychotic drug, such as thioridazine or fluphenazine, was effective in decreasing the growth of brain cancer cells and/or brain cancer stem cells.

It is believed that a person of ordinary knowledge in the art where the present invention belongs can utilize the present invention to its broadest scope based on the descriptions herein with no need of further illustration. Therefore, the descriptions and claims as provided should be understood as of demonstrative purpose instead of limitative in any way to the scope of the present invention.

The foregoing examples and description of the preferred embodiments should be taken as illustrating, rather than, as limiting the present invention, as defined by the claims. As will, be readily appreciated, numerous variations and combinations of the features set forth above can be utilized without, departing from the present invention as set forth in the claims. Such variations are not regarded as a departure from the scope of the invention, and all such variations are intended, to be included within the scope of the following claims. All references cited herein are incorporated herein in their entireties.

I/we claim:

1. A method for treating a brain tumor in a subject comprising administering to the subject in need thereof a therapeutically effective amount of an antipsychotic phenothiazine derivative.

2. The method of claim 1, wherein the brain tumor is a primary brain tumor.

3. The method of claim 1, wherein the brain tumor is a metastatic brain tumor.

4. The method of claim 1, wherein tire brain tumor is glioblastoma.

5. The method of claim 1, wherein the antipsychotic phenothiazine derivative is selected from the group consisting of thioridazine, acepromazine, fluphenazine, perphenazine, prochlorperazine, promazine, promethazine, trifluoperazine, triflupromazine and chlorpromazine.

6. The method of claim 5, wherein the antipsychotic phenothiazine derivative is thioridazine, fluphenazine, trifluoperazine or prochlorperazine.

7. The method of claim 1, wherein the antipsychotic phenothiazine derivative is thioridazine.

8. The method of claim 1, wherein the antipsychotic phenothiazine derivative is fluphenazine.

9. The method of claim 1, wherein die treatment is achieved by inhibiting the growth of brain, cancer cells and/or brain cancer stem cells.

10. A method, for inhibiting the growth, of brain cancer stem cells in a subject in need thereof comprising administering to the subject a therapeutically effective amount of an antipsychotic phenothiazine derivative.

11. The method of claim 10, wherein the brain cancer is glioblastoma.

12. The method of claim 10, wherein the antipsychotic phenothiazine derivative is selected from the group consisting of thioridazine, acepromazine, fluphenazine, perphenazine, prochlorperazine, promazine, promethazine, trifluoperazine, triflupromazine and chlorpromazine.

13. The method of claim 12, wherein the antipsychotic phenothiazine derivative is thioridazine, fluphenazine, trifluoperazine or prochlorperazine.

14. The method of claim 10, wherein the antipsychotic phenothiazine derivative is thioridazine.

15. The method of claim 10, wherein the antipsychotic phenothiazine derivative is fluphenazine.

16. A method for treating a brain tumor in a subject comprising administering to the subject in need thereof a therapeutically effective amount of an antipsychotic phenothiazine derivative in combination with another therapeutic drug for treating a brain tumor.

17. The method of claim 16, wherein the antipsychotic phenothiazine derivative is selected from the group consisting of thioridazine, acepromazine, fluphenazine, perphenazine, prochlorperazine, promazine, promethazine, trifluoperazine, triflupromazine and chlorpromazine.

18. The method of claim 17, wherein the antipsychotic phenothiazine derivative is thioridazine, fluphenazine, trifluoperazine or prochlorperazine.

19. The method of claim 16, wherein the antipsychotic phenothiazine derivative is thioridazine.

20. The method of claim 16, wherein the antipsychotic phenothiazine derivative is fluphenazine.
